SEQUENCE LISTING (6 sequences)

SEQ ID NO: 1 - DNA, 22 bp, Artificial Sequence
  Synthetic construct
aggacaagttgcatgagcagtg

SEQ ID NO: 2 - DNA, 19 bp, Artificial Sequence
  Synthetic construct
tcctggctggtgttggtag

SEQ ID NO: 3 - DNA, 20 bp, Artificial Sequence
  Synthetic construct
ctggctccttggctgagttc

SEQ ID NO: 4 - DNA, 18 bp, Artificial Sequence
  Synthetic construct
gcccagcctggttatcca

SEQ ID NO: 5 - DNA, 20 bp, Artificial Sequence
  Synthetic construct
cgtgacattaaggagaagct

SEQ ID NO: 6 - DNA, 18 bp, Artificial Sequence
  Synthetic construct
tcaggcagctcgtagctc

(19) United States
(12) Patent Application Publication
(10) Pub. No.: US 2015/0174273 A1
(43) Pub. Date: Jun. 25, 2015

(54) IMAGING AND THERAPEUTIC METHODS FOR TREATING PARATHYROID TUMORS

(71) Applicant: EMORY UNIVERSITY, Atlanta, GA (US)

(72) Inventor: Collin J. Weber, Atlanta, GA (US)

(73) Assignee: EMORY UNIVERSITY, Atlanta, GA (US)

(21) Appl. No.: 14/416,694

(22) PCT Filed: Jul. 24, 2013
(86) PCT No.: PCT/US13/51795

§ 371 Date: Jan. 23, 2015

Related U.S. Application Data
(60) Provisional application No. 61/675,367, filed on Jul. 25, 2012.

(51) Int. Cl. A61K 51/04

(52) U.S. Cl. CPC A61K 51/0459; A61K 51/0402

(57) ABSTRACT

It has been discovered that human parathyroid tumor cells express high densities of folate receptors which could provide a target that may be used for localization. In certain embodiments, the disclosure relates to methods of detecting and imaging parathyroid tumors or canccrous cells in tissues using a folatc conjugate to enhance imaging techniques such as magnetic resonance imaging, positron emission tomography, computed tomography (CT), and single-photon emission computed tomography (SPECT). An image of radioactivities or nuclear magnetic resonance frequencies as a function of location for parcels (voxels), may be constructed and plotted. The image shows the tissues in which the tracer has become concentrated.

### CROSS-REFERENCE TO RELATED APPLICATIONS

This application is a 371 USC of International Application No. PCT/US2013/051795 filed on Jul. 24, 2013, and claims the benefit of priority to U.S. Provisional Application No. 61/675,367 filed Jul. 25, 2012, which applications are hereby incorporated by reference in their entireties.

### BACKGROUND

Hyperparathyroidism is an increasingly significant medical and public health condition. In the past two decades, the incidence of hyperparathyroidism has increased 300%, and currently the disease affects at least 30,000 new patients each year in the United States. Parathyroid adenomas, parathyroid hyperplasia in primary and secondary hyperparathyroidism, and parathyroid carcinomas all are increasing in frequency. The mechanisms responsible for the increased incidence of hyperparathyroidism are not known. Environmental factors such as ionizing radiation exposure have been suggested by some authorities. Multiple organs are affected in patients with hyperparathyroidism; notably, a worsening of the severity of osteoporosis and accelerated arteriosclerotic disease and hypertension. Parathyroid carcinoma no longer is a rare illness, and there is no effective oncologic therapy for parathyroid carcinoma, which often is fatal. Thus, there is a need to identify improved therapies.

Surgery is the only effective management for primary hyperparathyroidism. Preoperative localization of the adenoma allows unilateral neck exploration for removal of the tumor. If localization is accurate, patients can undergo focal parathyroidectomies with cure rates equivalent to conventional surgery, less anesthesia, improved cosmesis, and a shorter hospital stay. Since this approach decreases both the duration of surgery and morbidity, preoperative localization is gaining recognition as an important procedure. However, tumor localization can be challenging, in part because current imaging methodologies are sub-optimal failing to identify the parathyroid tumor in as many as 30% of patients. In re-operative parathyroidectomy for persistent or recurrent hyperparathyroidism, localization plays an even greater role. Unfortunately, current multiple imaging modalities fail to localize 10-15% these of tumors. Thus, there is a need to identify improved methods of detection.

Positron Emission Tomography (PET) allows molecular imaging and is increasingly available throughout the US. PET/CT allows both functional/molecular imaging. ¹⁸F DG and C-11 methionine have been used to localize parathyroid adenomas with varying degrees of success. See Neumann et al. J Nucl Med 1996; 37:1809-1815 and Weber et al., Horm Metab Res 2010; 42(3):209-214. Being a well differentiated benign tumor, parathyroid adenomas have a low glucose metabolic rate and FDG uptake is moderate. C-11 methionine, a natural amino acid, is metabolized. In addition, C-11 has a short half life and requires a cyclotron for synthesis. Hence, C-11 has not been studied in large patient populations.

A technique for preoperative localization of human parathyroid tumors is a SPECT/CT, utilizing ^{99m}Tc sestamibi (MIBI) as a radiotracer. ^{99m}Tc MIBI early and delayed (dual phase) imaging with Single Photon Emission Computerized Tomography (SPECT) or SPECT/CT has become the standard of care. ^{99m}Tc MIBI is an isonitrile compound which tends to accumulate in mitochondria and has a short half life of 6 hours. These physical characteristics are suited for imaging with a Gamma camera. ^{99m}Tc MIBI concentrates both in thyroid and parathyroid tissues but washes out faster from thyroid tissue than parathyroid tumors, allowing dual phase imaging to localize the parathyroid tumors. SPECT imaging improves the contrast and facilitates location of the parathyroid tumors, while SPECT/CT provides three-dimensional localization. However, the reported sensitivity and specificity of ^{99m}Tc MIBI is only 80%. Parathyroid glands usually are located in close proximity to the thyroid and ^{99m}Tc MIBI concentrates both in thyroid and parathyroid tissue. Hence, there is a need for a tracer/imaging tool that concentrates in parathyroid cells more than in thyroid cells.

Folate receptors are found in some cancers. For example, pituitary ademomas provided differential expression of folate receptor. See Evans et al., Cancer Res 2003; 63:4218-4224. Folate receptor-targeted drugs are being developed for cancer and inflammatory diseases. Lu et al., Adv Drug Deliv Rev 2004; 56:1055-1058. Folate-receptors have been targeted with radionuclide imaging agents. See Ke et al., Adv Drug Deliv Rev 2004; 56:1143-1160.

### SUMMARY

It has been discovered that human parathyroid tumor cells express high densities of folate receptors which could provide a target that may be used for localization. In certain embodiments, the disclosure relates to methods of detecting and imaging parathyroid tumors or cancerous cells in tissues using a folate conjugate to enhance imaging techniques such as magnetic resonance imaging, positron emission tomography, computed tomography (CT), and single-photon emission computed tomography (SPECT). An image of radioactivities or nuclear magnetic resonance frequencies as a function of location for parcels (voxels), may be constructed and plotted. The image shows the tissues in which the tracer has become concentrated.

In certain embodiments, the disclosure relates to methods comprising a) administering a metal particle-folate conjugate to a subject at risk of, suspected of, or diagnosed with a parathyroid tumor; b) exposing an area suspected of containing the parathyroid tumor of the subject to a magnetic field and a radio frequency pulse; and c) detecting nuclear resonance frequencies in the area. The methods typically further comprise the step of creating an image from the detected nuclear resonance frequencies. The metal particle is typically an iron oxide nanoparticle.

In certain embodiments, the disclosure relates to methods comprising a) administering a radioisotope-folate conjugate to a subject at risk of, suspected of, or diagnosed with a parathyroid tumor, and b) detecting gamma rays in an area of the subject. The methods typically further comprise the step of creating an image from the detected gamma rays. An example of a radioisotope is 99mTechnetium, and a radioisotope-folate conjugate is Folatescan, ^{99m}Tc-EC20, Endocyte, Inc.

In certain embodiments, the disclosure relates to methods comprising a) administering a composition comprising a positron-emitting radionuclide or a radionuclide-folate conjugate to a subject at risk of, suspected of, or diagnosed with a parathyroid tumor, and b) detecting photons moving in approximately opposite directions in an area of the subject. Typically the methods further comprising creating an image from the detected photons. An example of a positron-emitting radionuclide is anti-1-amino-[¹⁸F]flurocyclobutane-1-carboxylic acid (anti-¹⁸F-FACBC). In certain embodiments, the disclosure relates to a folate conjugate comprising a positron-emitting radionuclide and uses for imaging.

Within certain embodiments, the disclosure contemplates using methods disclosed herein to detect parathyroid cancer including metastasized cancer and further administering a chemotherapeutic agent or removing cancerous cells by surgery based information obtain from the imaging technique.

In certain embodiments, the disclosure contemplates treating PT cancer comprising administering an effective amount of a pharmaceutical composition comprising a folate anticancer drug conjugate to a subject in need thereof. In certain embodiments, a subject is diagnosed with, exhibiting symptoms of, or at risk of cancer

### BRIEF DESCRIPTION OF THE FIGURES

FIG. 1A show data on experiments for FR expression in PT by IHC. and normal PT showing strong and diffuse staining for the FR by IHC (black arrows). The surrounding normal thyroid follicles (blue arrows) are negative.

FIG. 1B is the high-power photomicrograph of a PT adenoma showing both membrane and cytoplasmic staining for the FR.

FIG. 1C is infiltrative PT carcinoma with positive immunoreactivity for FR with no staining noted in the surrounding stroma.

FIG. 1D is PT 2° hyperplasia staining positive for FR.

FIG. 1E is adenomatiod thyroid nodule composed of large follicles distended with colloid; the flattened follicular epithelium is negative for FR.

FIG. 2 shows data on experiments of FRα expression by normal human PT and renal failure hyperplasias by Western blotting. PT tissue homogenates (60 μg) and HeLa, KB, and Jurkat cell lysates (20 μg) were separated by gel (12%) electrophorsis under non-reducing conditions and transferred to a polyvinylidene difluoride (PVDF) membrane. The membrane was blocked to prevent any nonspecific binding of antibodies to the surface of the membrane, and FRα was detected with a primary antibody (Ab 343), followed by staining with a secondary goat anti-mouse IgG antibody conjugated to alkaline phosphatase (1:1000). Molecular weight markers (20 to 250 kDa) were included as standards. PT tissue included samples from 2 normal PT glands and from 2 patients (#1 and #2) with tertiary (3o) hyperplasia. RU=right upper PT gland, LU=left upper PT gland.

FIG. 3 shows data on the assessment of FRα (FolR1) and FRβ (FolR2) expression in human PT renal failure hyperplasia specimens by quantitative RT-PCR. Total RNA was extracted from three human PT hyperplasia samples and from control Jurkat cells (FR negative) using RNeasy Mini Kits (Qiagen). RNA was quantified by spectro-photometry, and equivalent amounts (950 ng) of RNA were used for cDNA synthesis using random nonamers. The RT products (0.2 uL) were used in PCR and in qPCR (SYBR green method), with primers for the FRα (FolR1) and beta-actin primers as endogenous controls. The Y axis shows the relative quantification of the m-RNA levels of FolR1 and 2 in different parathyroid tissues taking Jurkat cells as the reference and beta-actin as the endogenous control. HP=hyperplasia; RU=right upper parathyroid; LU=left upper parathyroid; #1=patient one; #2=patient 2.

FIG. 4 shows data from experiments to target specificity of 99mTc(CO)3-folate in PT and thyroid cells. Two different doses of human PT adenoma cells and thyroid cells (10 μL [blue bars] and 20 μL tissue [red bars]) were incubated with 99mTc(CO)3-folate, as described in the Methods section, and the dose uptake of 99mTc(CO)3-folate was assessed using a gamma counter. The amount of 99mTc(CO)3-folate incorporated by each group was reported as the mean±standard deviation (SD). *=Significantly higher incorporation by 20 μl PT tissue compared to 10 μl PT tissue, p<0.05 by ANOVA; **=Significantly higher 99mTc(CO)3-folate incorporated by 10 μl PT adenoma vs. 10 μl thyroid, p<0.05, by ANOVA; 1′ Significantly higher 99mTc(CO)3-folate incorporated by 20 μl PT adenoma vs. 20 μl thyroid, p<0.001, by ANOVA)

FIG. 5 shows data on dose-dependent uptake of 99mTc-EC20 (a folate-derived 99mTc-based radiopharmaceutical) (blue bars) by a slurry of freshly-excised, non-cultured human parathyroid adenoma cells. Some aliquots of cells were blocked by pre-incubation with cold folate (FA) (yellow bars). The amount of 99mTc-EC20 incorporated by each group was reported as the mean±standard deviation (SD). *=Significantly higher incorporation in the absence (blue bars) compared to the presence of cold folate (yellow bars), 20 μl dose, <0.05 by ANOVA; **=Significantly higher incorporation in the absence (blue bars) compared to the presence of cold folate (yellow bars), 70 μl dose, <0.001 by ANOVA.

FIG. 6 shows an illustration of folate ligands for the preparation of 99m Tc(CO)₃-folate or other traceable metal isotopes such as ^{99m}Tc or ¹⁸⁸Re.

FIG. 7 shows data from an ¹⁸F FACBC uptake assay. BCH is L type transporter inhibitor (2-amino-2-norboranecarboxylic acid), MeAlB is an A type inhibitor (2-[methylamine]isobutric acid), ACS is a multiple amino acid transporter inhibitor (L-alanine, L-cystine, L-serine).

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims. Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

Prior to describing the various embodiments, the following definitions are provided and should be used unless otherwise indicated.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein the term “folate conjugate” refers to a molecule containing a 4-(((2-amino-4-oxo-3,4,4a,8a-tetrahydropteridin-6-yl)methyl)amino)benzamide moiety sufficient for binding a folate receptor.

### Expression of Functional Folate Receptors by Human Parathyroid Cells

Parathyroid (PT) cancer has no known effective therapy once metastasized. A systemic therapy to control the metastatic parathyroid cancer is needed. A more sensitive and specific radiotracer/tracking agent would markedly improve identification of parathyroid tumors preoperatively and localization of tumors intra-operatively, and thus offer more patients a minimally invasive parathyroidectomy, while reducing healthcare costs.

It was explored whether PTs (including normal, hyperplastic and neoplastic) expressed folate receptors (FR). In addition, normal thyroid also was evaluated for FR expression. Since one of the aims was to find a more useful imaging technology to localize PTs, it would be important to be able to distinguish parathyroids from thyroids. Importantly, experiments were performed to determine whether FRs are functional on human PTs, and whether ligands such as 99m Tc(CO)₃-folate and 99mTc-etarfolatide (99mTc-EC20), have affinity for FR positive cells, with specific dose-responsive activity in vitro.

Another aim of experiments herein were to evaluate FR expression in PT cancer (PT cancer). The FR has been investigated as a potential for tumor-specific therapy. Several human tumors have been shown to over-express FR, including tumors of the breast, colon, ovary, and uterus. About 30% of squamous cell carcinomas from the larynx and oral cavity express FR. The success of targeted therapy is dependent on uniform and strong expression of the FR. The etiology of PT CA is unknown, with rare reports of PT CA arising in long-standing secondary hyperparathyroidism or in patients with a history of irradiation for the neck. PT CA has a high morbidity associated with severe hypercalcemia. Recurrences range from 25-80% after initial surgery and 25% of patients develop distant metastases. Due to the paucity of chemotherapy treatment options for this neoplasm, it would be highly desirable to identify new treatment strategies, including targeted therapy. Drugs that target FR, resulting in enhanced drug delivery, will likely improve the overall survival of patients with this disease.

Experiments herein indicate that FR expression in normal human PT, PT adenomas, PT hyperplasias and PT carcinomas. Two PT carcinomas were available for in vitro study, and five archival carcinomas were available for IHC. These findings indicate the use of radiolabeled folate for identification and localization of PT tumors, both pre-operatively and intra-operatively. PT tumors may be imaged using 99mTc-MIBI, which detects both PT tumors and normal thyroids. Experiments herein indicate that PT cells can be imaged specifically with a labeled folate tracer that will target FR positive PTs but not adjacent thyroid glands, which lack FR expression. We believe that the absence of folate binding by thyroid tissue adjacent to PT tumors offers a significant advantage for the use of radiolabeled folate over MIBI, wherein false positive thyroid nodules and tumors often interfere with accurate PT tumor identification.

In addition, the relatively strong expression of FRα in hyperplasias suggests to us that targeting PT hyperplasias with a radiolabeled folate probe could be far superior to conventional imaging with 99m Tc-MIBI, since 99m Tc-MIBI rarely visualizes hyperplasias. Furthermore, since 99mTc-MIBI visualizes only 70%-90% of adenomas, a radiolabeled folate tracer may be superior to 99mTc-MIBI for imaging adenomas, as well.

Adequate radioimaging does not require FR saturation. In fact, only 100 μg of 99mTc-EC20 per patient is needed, which translates to an approximate initial serum concentration (Ci) of ˜60 nM if one assumes that i) blood is 7% total body weight, ii) average hematocrit of 45%, and iii) 70 kg patient.

Freshly resected, viable human PT cells have folate binding activity indicate the functionality of this receptor for use of folate-drug conjugates or folate-based radionuclide imaging and therapy for PT neoplasms. Folate conjugation to anti-cancer drugs are useful to deliver therapeutic agents selectively to PT CA because folate binds to the FR and is internalized by receptor-mediated endocytosis. As FR expression is restricted in most normal tissues, developing a folate-targeted cytotoxic drug is useful for the treatment of PT CA.

### Imaging and Therapy

In certain embodiments, this disclosure contemplates methods of imaging using folate-conjugated SPIO nanoparticles. Superparamagnetic iron oxide (SPIO) nanoparticles are typically less than 50 nm in diameter made up of an iron oxide core stabilized by an organic shell. Human parathyroid tumors are thought to express folate receptors. SPIO nanoparticles can be labeled with fluorescence or radioactivity and targeted to specific ligands, such as the folate receptor. See Peng et al., Int J Nanomedicine. 2008; 3(3): 311-321 and Sonvico et al., Bioconjug Chem. 2005; 16(5):1181-8, and Sun et al., Biomed Mater Res A. 2006; 78(3):550-7, and Chen et al., PDA J Pharm Sci Technol. 2007; 61(4):303-13, all hereby incorporated by reference.

An MRI (Magnetic Resonance Imaging) scanner typically consists of magnet of 1.5 to 7, or more Tesla strength. A magnetic field and radio waves are used to excite protons in the body. These protons relax after excitation, and a computer program translates this data into pictures of human tissue. In certain embodiments, this disclosure contemplates that a pre-contrast image is taken. Once the SPIO nanoparticles are injected, a post-contrast image is taken. A contrast is detected wherever the nanoparticles aggregate in the body.

In certain embodiments, this disclosure contemplates methods of imaging using 99mTc-folate. The in-vivo diagnosis of tumor receptor expression allows selection of tumors that may be treatable by targeted therapy such as a folate-drug conjugate or folate-based radionuclide therapy. Normal tissues that lack folate receptors could be spared toxicity associated with non-targeted drug delivery. Folate-based imaging agents, including radiopharmaceuticals, may provide diagnostic testing by locating and assessing the receptor density of folate receptor-positive tumors.

Several labeled folate conjugates are contemplated including ^{99m}Tc, ⁶⁷Gallium, and ¹¹¹In DTPA conjugates. See Mathias et al., J Nucl Med 1996; 37:1003-1008 and Wang et al., Bioconjug Chem 1997; 8:673-679, hereby incorporated by reference. Chelators may be used to label ^{99m}Tc with folate. See Ke et al., Adv Drug Deliv Rev 2004; 56:1143-1160 and Trump et al., Nucl Med Biol 2002; 29:569-573, and Müller et al., Nucl Med Biol 2007; 34:595-601, all hereby incorporated by reference.

In certain embodiments, the disclosure contemplates imaging and therapy on metastatic parathyroid cancer. A gamma emitter such as ^{99m}Tc may be used for a diagnostic probe. A beta minus emitters can be a therapeutic. In certain embodiments, it is contemplated that Na I-123 (a gamma emitter) is used for diagnosis and localization of parathyroid cancer metastases and Na I-131 (a beta minus emitter) is used for therapy. ^{99m}Tc and ¹⁸⁸Re-rhenium (¹⁸⁸Re) are an attractive pair of radionuclides for biomedical use, because of their favorable decay properties for diagnosis (^{99m}Tc: 6 hour half-life, 140-keV γ-radiation) and therapy (¹⁸⁸Re:17 hour half-life, 2.12-MeV β-maximum-radiation). Thus, certain embodiments of the disclosure contemplate simultaneous diagnostic and therapeutic methods within the same compositions for the management of metastatic parathyroid cancer, e.g., using ^{99m}Tc-Folate and ¹⁸⁸Re-Folate conjugates.

In certain embodiments, the disclosure contemplates methods of ¹⁸F-FACBC Imaging. Anti-18 F-FACBC (anti-1-amino-¹⁸F-flurocyclobutane-1 carboxylic acid) is a non-natural amino acid and is an L-leucine analog with low renal excretion and high pancreatic concentration. See McConathy et al., Appl Radiat Isot. 2003; 58(6):657-66, hereby incorporated by reference. Parathormone is a peptide hormone, and the bioactive conformation includes a long helical dimer containing leucine residues. In preliminary experiments, primary human parathyroid cells exhibited significant specific uptake of Anti-¹⁸F-FACBC. It is contemplated that parathyroid cells concentrate Anti-¹⁸F-FACBC, and thus Anti-¹⁸F-FACBC can be used as an imaging probe for PET imaging.

Nuclear Magnetic Resonance (NMR) and Magnetic Resonance Imaging (MRI) are techniques for identifying isotopes in a sample (area) by subjecting the sample to an external magnetic fields and detecting the resonance frequencies of the nuclei. NMR typically involves the steps of alignment (polarization) of the magnetic nuclear spins in an applied, constant magnetic field and perturbation of this alignment of the nuclear spins by employing an electro-magnetic radiation, usually radio frequency (RF) pulse. A pulse of a given carrier frequency contains a range of frequencies centered about the carrier frequency. The Fourier transform of an approximately square wave contains contributions from the frequencies in the neighborhood of the principal frequency. The range of the NMR frequencies allows one to use millisecond to microsecond radio frequency pulses.

Resonant absorption by nuclear spins will occur when electromagnetic radiation of the correct frequency is being applied to match the energy difference between the nuclear spin levels in a constant magnetic field of the appropriate strength. Such magnetic resonance frequencies typically correspond to the radio frequency (or RF) range of the electromagnetic spectrum for magnetic fields. It is this magnetic resonant absorption which is detected. In Magnetic Resonance Imaging (MRI), detected frequencies of atoms are typically used to create images. Hydrogen is the most frequently imaged nucleus in MRI because it is present in biological tissues in great abundance. However, any nucleus with a net nuclear spin could potentially be imaged with MRI.

Single-photon emission computed tomography (SPECT) is an imaging technique using gamma rays. Using a gamma camera, detection information is typically presented as cross-sectional slices and can be reformatted or manipulated as required. One injects a gamma-emitting radioisotope (radionuclide) into a subject. The radioisotope contains or is conjugated to a molecule that has desirable properties, e.g., a marker radioisotope has been attached to a ligand, folate, which is of interest for its chemical binding properties to certain types of tissues. This allows the combination of ligand, e.g., folate, and radioisotope (the radiopharmaceutical) to be carried and bound to a place of interest in the body, which then (due to the gamma-emission of the isotope) allows the ligand concentration to be seen by a gamma-camera.

Positron emission tomography (PET) is an imaging technique that produces a three-dimensional image. The system detects pairs of gamma rays emitted indirectly by a positron-emitting radionuclide (tracer). Three-dimensional images of tracer concentration within the area are then constructed by computer analysis. A radioactive tracer isotope is injected into subject e.g., into blood circulation. Typically there is a waiting period while tracer becomes concentrated in tissues of interest; then the subject is placed in the imaging scanner. As the radioisotope undergoes positron emission decay, it emits a positron, an antiparticle of the electron with opposite charge, until it decelerates to a point where it can interact with an electron, producing a pair of (gamma) photons moving in approximately opposite directions. These are detected in the scanning device. The technique depends on simultaneous or coincident detection of the pair of photons moving in approximately opposite direction (the scanner has a built-in slight direction-error tolerance). Photons that do not arrive in pairs (i.e. within a timing-window) are ignored. One localizes the source of the photons along a straight line of coincidence (also called the line of response, or LOR). This data is used to generate an image.

Within any of the imaging embodiments, methods disclosed herein may further comprise the steps of recording the images from an area of the subject on a computer or computer readable medium. In certain embodiments, the methods may further comprise transferring the recorded images to a medical professional representing the subject under evaluation.

In certain embodiments, the disclosure contemplates treating PT cancer comprising administering an effective amount of a pharmaceutical composition comprising a folate anticancer drug conjugate to a subject in need thereof. In certain embodiments, a subject is diagnosed with, exhibiting symptoms of, or at risk of cancer. In certain embodiments, the folate anti-cancer conjugate comprises the anticancer drug selected from gefitinib, erlotinib, docetaxel, cis-platin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin and mithramycin, vincristine, vinblastine, vindesine, vinorelbine taxol, taxotere, etoposide, teniposide, amsacrine, topotecan, camptothecin, bortezomib, anagrelide, tamoxifen, toremifene, raloxifene, droloxifene, iodoxyfene, fulvestrant, bicalutamide, flutamide, nilutamide, cyproterone, goserelin, leuprorelin, buserelin, megestrol, anastrozole, letrozole, vorazole, exemestane, finasteride, marimastat, trastuzumab, cetuximab, dasatinib, imatinib, bevacizumab, combretastatin, thalidomide, and/or lenalidomide or combinations thereof.

### EXAMPLES

### PT and Thyroid Samples

With Institutional Review Board (IRB) approval, formalin-fixed paraffin embedded archival PT tissues from the files in the Department of Pathology, Emory University Hospital were identified: 21 PT adenomas (2 sestamibi negative), 9 primary hyperplasia, 13 secondary hyperplasia (end-stage renal disease; 2 sestamibi negative), 5 PT CA, and 9 normal PTs. In addition, normal adjacent thyroid, 3 thyroid medullary carcinomas and 4 adenomatoid thyroid nodules were evaluated. Fresh operative PT tissue included portions of 33 resected PT tumors and 6 samples of normal thyroid tissue obtained from patients with IRB approval. For collection of normal PT cells, these glands were routinely dissected from the surface of the thyroid goiters and tumors, minced finely in a Petri dish, and returned to the patients as autografted PT fragments. Afterwards, small numbers of residual normal PT cells left in the Petri dish that would have been discarded were suspended in HBSS for study.

### Cell Cultures

FRα positive cell lines, KB (ATCC# CCL-17, subline of HeLa) and HeLa (ATCC # CCL-2, human epithelial cervical cancer), FRβ positive cells (Chinese hamster ovary [CHO] cells expressing FRβ) and FR negative cell lines, A549 (adenocarcinomic human alveolar basal epithelial cells) and Jurkat (ATCC # TIB-152, a human T cell lymphoblast-like cell line) were cultured as monolayers at 37° C. in a humidified atmosphere containing 5.0% CO₂. Fresh, human PT and thyroid glands were minced, washed twice with Hanks's balanced salt solution (HBSS) and incubated in 2 mg/ml collagenase (CLS4, Type 4, Worthington Biochemical Corp., Lakewood, N.J., USA) or endotoxin-free liberase (Roche Diagnostics Corp., Indianapolis, Ind., USA) for 1-1.5 h in a 37° C. shaking water bath (170 rpm) with vigorous hand shaking at 30-min intervals.

The dissociated cells were passed through sterile nylon mesh (500 μM), washed in HBSS and resuspended in RPMI-1640 (0.45 mM/1 calcium, 0.4 mM/1 magnesium) plus 10% fetal bovine serum (FBS), 2 mM L-glutamine, 10 mM Hepes, 0.5 mM Na pyruvate, 100 IU/ml penicillin and 100 μg/ml streptomycin. The cells were plated at 0.5-1×10⁶/ml in 12-well dishes and cultured for 1-9 days at 37° C., 5.0% humidified CO₂. At least 3 days before an experiment, all cells were transferred to folate-free (FFR) RPMI medium (Gibco, Life Technologies), supplemented with 10% heat-inactivated fetal calf serum (FCS), as the only source of folate), L-glutamine and antibiotics (penicillin 100 IU/ml, streptomycin 100 μg/ml) which has a final folate concentration of ˜3 nM, a value at the low end of the physiological concentration in human serum.

### FR Expression in PT Tissue by IHC

Immunohistochemical staining was performed using an avidin-biotin-peroxidase complex technique and steam heat-induced antigen retrieval, according to standard techniques. For negative controls, the specific antibody was replaced with buffer. FR expression in tissue specimens was analyzed using a goat anti-human FR polyclonal antibody (sc-16387, 1:100 dilution; Santa Cruz Biotechnology, Santa Cruz, Calif.).

All tissue sections were evaluated by a single pathologist (SM). The level of FR expression was considered positive when characteristic cytoplasmic or membranous staining was present. When present, normal thyroid tissue was also evaluated and graded. A scoring system reported by us was adopted: 0 score for no staining; 1+ for <25% of cells showing immunoreactivity; and 2+ for >25% of cells showing immunoreactivity (16). Expression of genes for FRα and FRβ in human PT tissue using Illumina Human HT-12 Expression

### Bead Chips

Analysis of FRα, FRβ, and FRγ gene expression was performed by the Emory Genomics Core Lab in the Winship Cancer Center. Briefly, total RNA was isolated from human PT tumor samples (1-5×10⁶ cells/sample) using RNEasy kits (Qiagen), and then Illumina Human HT-12 Expression Bead Chips were used, according to manufacturer's directions, and the data was analyzed by Ingenuity Pathway Analysis (Ingenuity Systems).

### Evaluation of FR Expression in PT Tissue by Western Blot

FR expression in normal PT samples, adenomas, and hyperplasias was determined. PT tissues were homogenized in Tris buffer with Triton X-100 and a cocktail of protease inhibitors. The homogenates were sonicated and centrifuged at 10,000 RPM (4° C.) for 10 to 15 minutes, and the supernatants were used for the Westerns blots. The KB, HeLa, and Jurkat cells were prepared as described above, except they were not homogenized and sonicated. PT tissue homogenates (60 μg) and HeLa, KB, and Jurkat cell lysates (20 μg) were separated by gel (12%) electrophoresis under non-reducing conditions and molecular weight markers (20 to 250 kDa) were included as standards. The separated proteins were transferred to a polyvinylidene difluoride (PVDF) membrane.

The membrane was blocked to prevent any nonspecific binding of antibodies to the surface of the membrane, and FRα was detected with a primary antibody (mAb 343, the kind gift of Dr. Phil Low), followed by staining with a secondary goat anti-mouse IgG antibody conjugated to alkaline phosphatase (1:1000). Bands were developed using an AP substrate kit (Biorad).

Relative Quantification of FRα and FRβ m-RNA Expression

Total RNA was extracted from various PT tissues, Jurkat, HeLa and CHO cells stably expressing FRβ using the RNeasy Mini Kit from Qiagen following the manufacturer's protocol. A total of 900 ng of total RNA was reverse transcribed using random nonamers and the enhanced Avian RT first strand synthesis kit (Sigma). Real-time PCR was performed for quantifying FRα and FRβ, as well as β-actin as the endogenous control for each sample. All amplifications were run in triplicates using express SYBR green ER kit (Invitrogen) on an Applied Biosystems StepOne Plus real time cycler. The amplification protocol used 0.2 μl of the transcribed cDNA, 0.2 μM of each primer, an initial denaturation at 95° C. for 5 minutes, followed by 40 cycles of 95° C. for 15 seconds, 600 C for 30 seconds followed by a melt curve to verify the specificity of the amplification. The primers used were:

FRα (FolR1) sense 5′-AGGACAAGTTGCATGAGCAGTG-3′ (SEQ ID NO:1) and antisense 5′-TCCTGGCTGGTGTTGGTAG-3′ (SEQ ID NO:2);

FRβ (FolR2) sense 5′-CTGGCTCCTTGGCTGAGTTC-3′ (SEQ ID NO:3) and anti-sense 5′-GCCCAGCCTGGTTATCCA3′ (SEQ ID NO:4); and β-actin sense 5′-CGTGACATTAAGGAGAAGCT-3′ (SEQ ID NO:5) and anti-sense 5′-TCAGGCAGCTCGTAGCTC-3′ (SEQ ID NO:6).

Results of the amplification are expressed relative to the analysis of the Jurkat negative control (=1) as the log value of the expression fold expansion.

### In Vitro Folate-Binding Experiments

The binding of 99mTc(CO)₃-folate by PT tumor cells versus thyroid cells was determined by incubating single-cell suspensions of thyroid and PT tumors with 99m Tc(CO)3-folate. In this study, 99mTc(CO)₃-folate was prepared at Emory University as described in Müller et al., Organometallic 99mTc-technetium(I)- and Re-rhenium(I)-folate derivatives for potential use in nuclear medicine. J Organomet Chem, 2004, 689:4712-21, utilizing the folate derivative, PAMA-γ-folate. Non-trypsinized, homogenized human thyroid and PT tumor cells were incubated with 99mTc(CO)₃-folate for 30 min at 37° C. (5% CO2/78% RH). After washing two times with PBS buffer, the percent dose uptake of 99mTc(CO)₃-folate was assessed using a gamma counter. The specific targeting of FRs on PT cells was demonstrated by blocking the binding of 99mTc-EC20 with cold folate. 99mTc-EC20, a folate-derived 99mTc-based radiopharmaceutical, was synthesized at Emory University, as described in Leamon et al., Synthesis and biological evaluation of EC20: A new folate-derived, 99mTc-based radiopharmaceutical, Bioconjugate Chem, 2002, 13:1200-10, using an EC20 kit. Increasing amounts of a slurry of PT adenoma cells (10 μl, 20 μl, and 70 μl) were incubated in triplicate with 99mTc-EC20 (˜6 uCi per assay tube) in the presence or absence of cold folate solution (200 μM). The dose-dependent uptake of the radio-labeled compound was measured by gamma counting.

### PT Tumor Cells, but not Normal Thyroids, are Positive for FR by IHC.

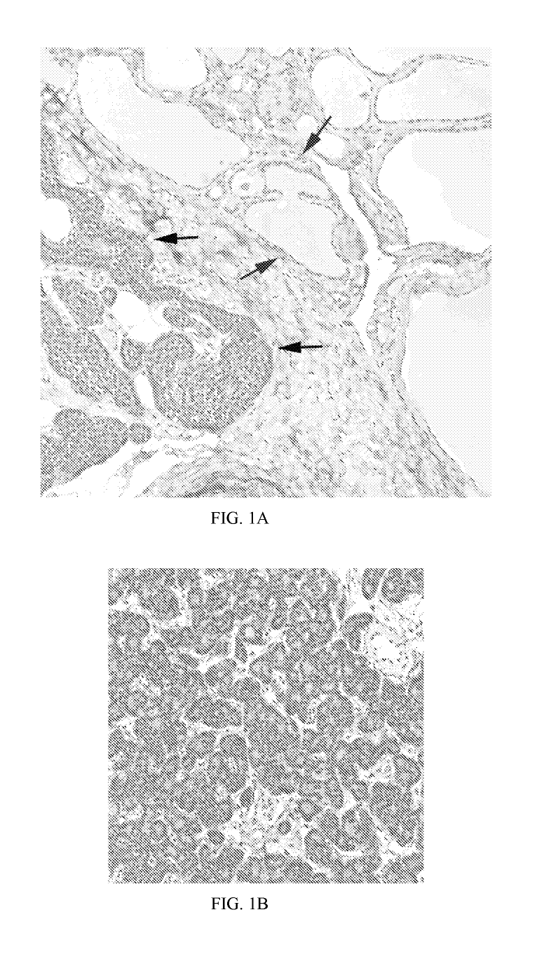

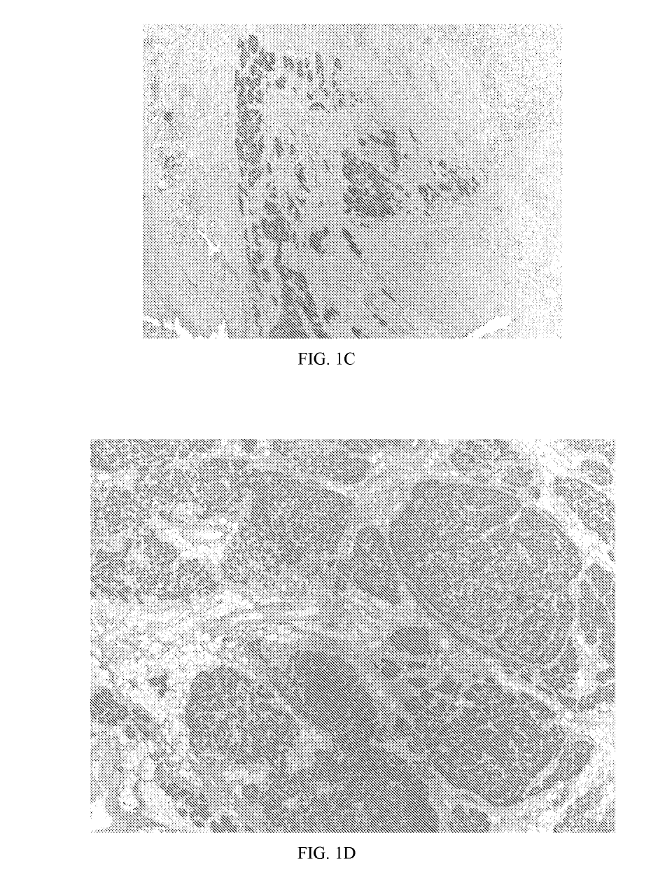

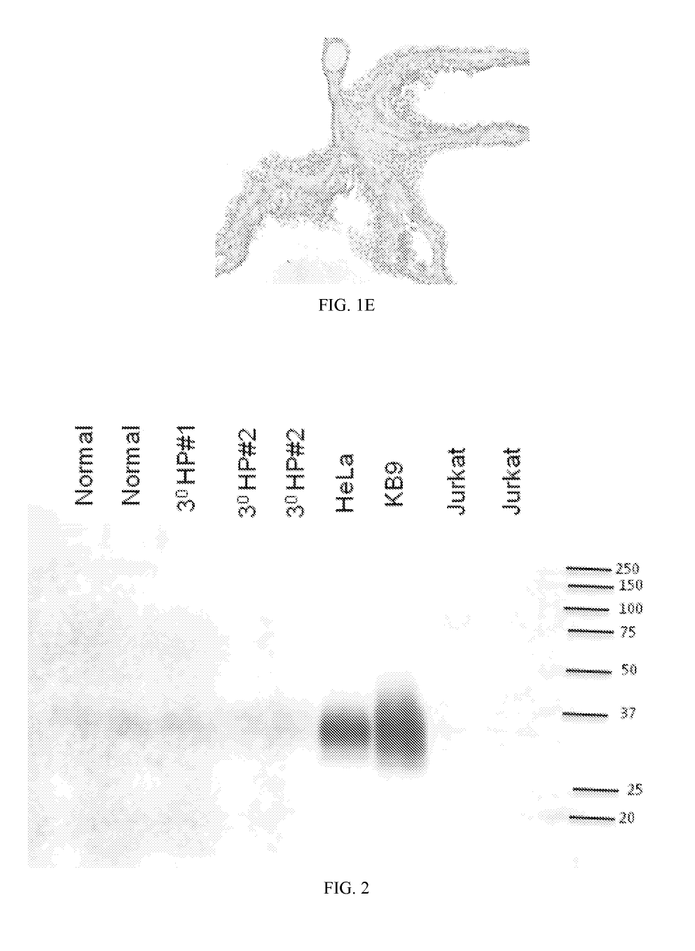

All tissue samples from patients with PT proliferative disorders and all normal PTs and showed strong and diffuse cytoplasmic and membranous immunoreactivity for FR (FIGS. 1A, 1B, 1C, and 1D). Both cytoplasmic and membrane staining were noted in the PT tumor cells. No qualitative or quantitative differences were seen, as in all cases the FR expression was strong, including the cases of secondary PT hyperplasias (FIG. 1D). None of the thyroid tissues, including adjacent normal thyroid (FIG. 1A) and thyroid neoplasms (FIG. 1E) were positive for FR by IHC. Head and neck cancer biopsies served as positive controls, and negative controls lacking the secondary antibody were negative.

### FRα and FR β Genes are Expressed in Human PTs.

Four isoforms of the FR family have been identified, i.e. FR α, β, δ, and γ. The α isoform of the FR is present on the apical surfaces of epithelial cells and is over-expressed in approximately 40% of human cancers (breast, lung, ovarian, uterine cancers, and head and neck squamous cell carcinomas). The β isoform is expressed in hematopoietic cells of the myelogenous lineage (11). Using Illumina Human HT-12 Expression Bead Chips, it was determined that the FR α gene was expressed in all PT samples studied, the FR β gene was expressed at lower levels by most samples, and the FRγ gene was not detected in normal PTs or hyperplasias (Table 1).

TABLE 1
Relative signal intensity detecting expression
of the genes for FRs α, β, and γ.

| | Normal PT | Adenoma | Hyperplasia |
|---|---|---|---|
| Gene | (n = 4) | (n = 4) | (n = 4) |
| FR α | 231.3 ± 25.6 | 194.7 ± 123.7 | 214.3 ± 57.5 |
| FR β | 49.6, 81.7* | 51.0, 57.7, 61.8* | 56.7 ± 9.5* |
| FR γ | Not detected | 46, 0, 47.5 ** | Not detected |

*FRβ was expressed by 2 of 4 normal PTs, 3 of 4 adenomas, and 4 of 4 hyperplasias.
** FRγ was expressed by 2 of 4 adenomas, but not by normal PTs or hyperplasias.

The FRα protein expression was documented in normal human PT and in PT tumors by Western blot. FRα expression was determined in normal PT and in PT hyperplasia specimens by Western blotting according to standard techniques, using a mouse anti-human FRα antibody (Ab 343) (FIG. 2). Positive controls included HeLa and KB cells; Jurkat cells served as negative controls. A 37 kDa band (FRα) was strongly detected in the HeLa and KB cell lysates, but no band was detected in Jurkat cell lysates. Weaker, but detectable, 37 kDa bands were present in tissue homogenates from normal PT and from 3o hyperplasia (FIG. 2), showing that FRα is expressed in normal human PT and in PT hyperplasias. Additional Western blots provided evidence that human PT adenomas also express FR α.

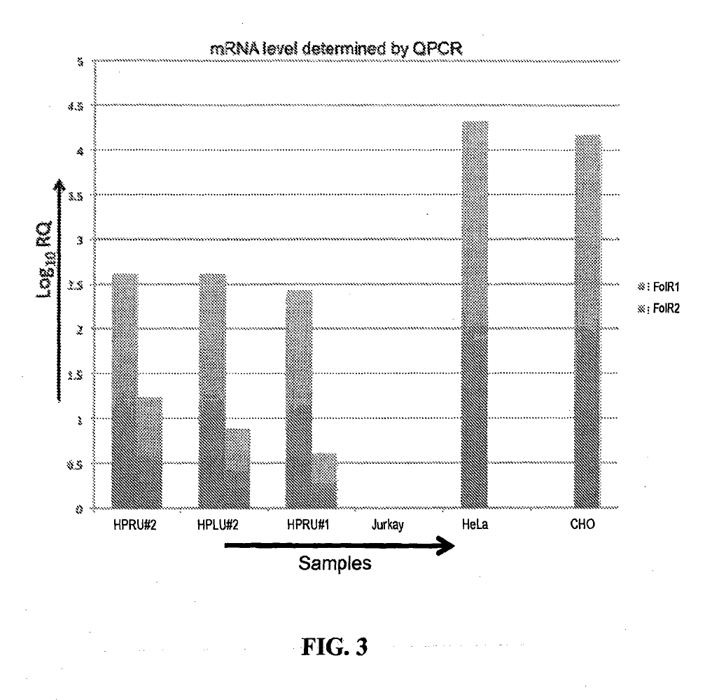

Relatively higher expression of FRα than FRβ was found by quantitative RT-PCR. To confirm the levels of FRα and FRβ expression in human PT tumors, quantitative RT-PCR was performed, using total RNA isolated from PT tissue homogenates. Human PT hyperplasias expressed FRα (FolR1) at levels of 2.4 to 2.6 Log 10RQ, and FRβ (FolR2) was expressed at lower levels (0.6-1.2 Log 10RQ), echoing the relative gene expression levels detected for these two isoforms of the FR in the microarray studies (FIG. 3). In the positive control HeLa cells, FRα levels were relatively high (>4 Log 10RQ), and in the positive control CHO cells transfected with FRβ, FRβ levels were equally high (>4 Log 10RQ). In negative control Jurkat cells, no amplification of FRα or FRβ was detected (FIG. 3).

### Demonstration of FR Functionality by In Vitro Folate-Binding Experiments.

To determine whether the FRs detected in our human PT samples are functional, folate-binding experiments were performed. Human PT tumor cells incorporate significantly more 99mTcfolate than thyroid cells. The amount of 99mTc(CO)3folate incorporated by PT adenoma cells versus thyroid cells was determined by incubating different doses of single-cell suspensions of PT adenomas and thyroids (10 uL and 20 uL samples) with 99mTc(CO)3folate, and uptake was determined by gamma counting.

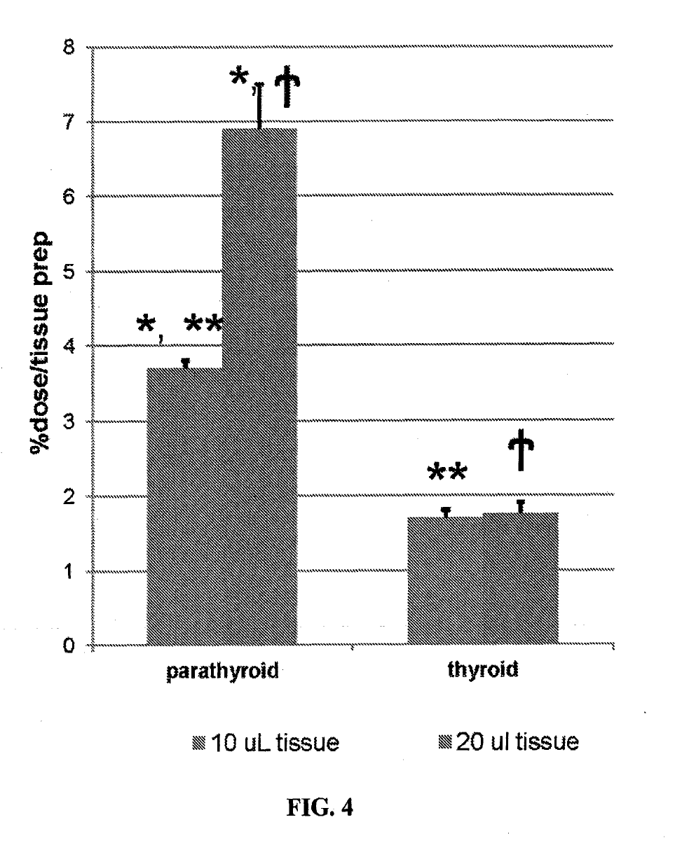

Significantly more 99mTc(CO)3-folate was incorporated by the higher dose of PT adenoma cells compared to the lower dose (p<0.05 by ANOVA), but no dose-dependent incorporation was seen in the thyroid cells (FIG. 4). There was significantly more uptake of 99mTc(CO)3folate by PT adenoma cells (in both the 10 uL and 20 uL tissue samples) when compared to thyroid (FIG. 4) (for example, 6.9±0.6 for PT adenoma vs 1.7±0.1 for thyroid, % dose/20 μl tissue sample, p<0.001 by ANOVA). These results suggest that PT adenomas express significantly more FR than thyroid cells, in agreement with our IHC analysis.

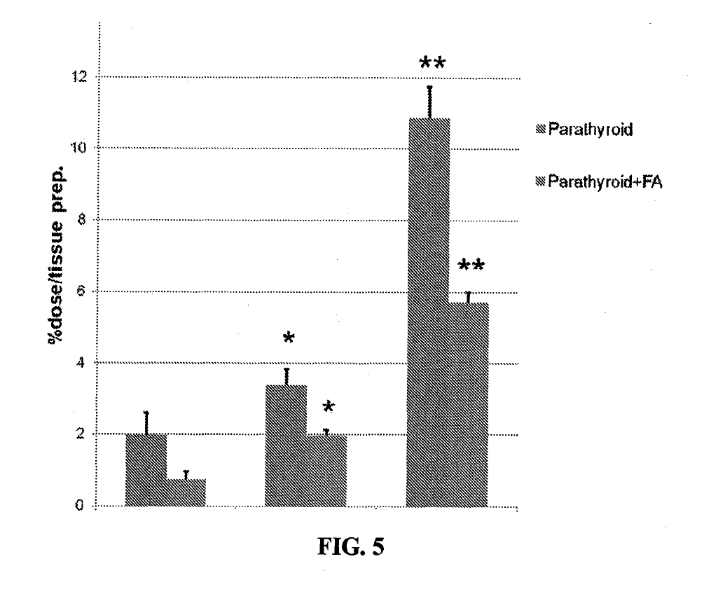

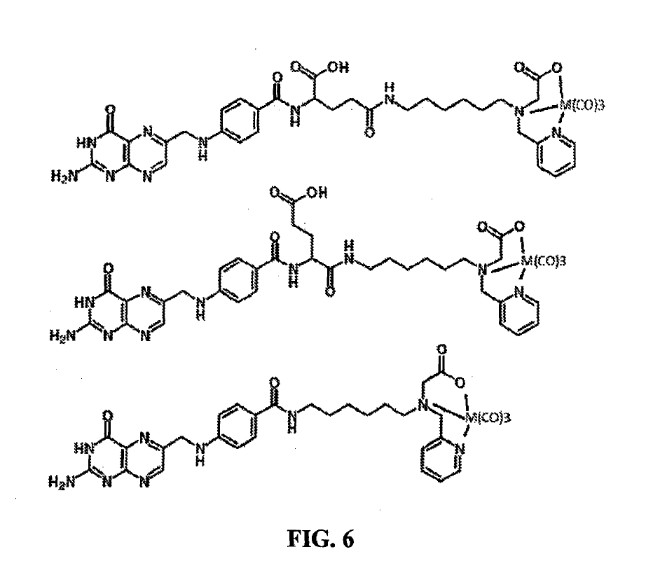

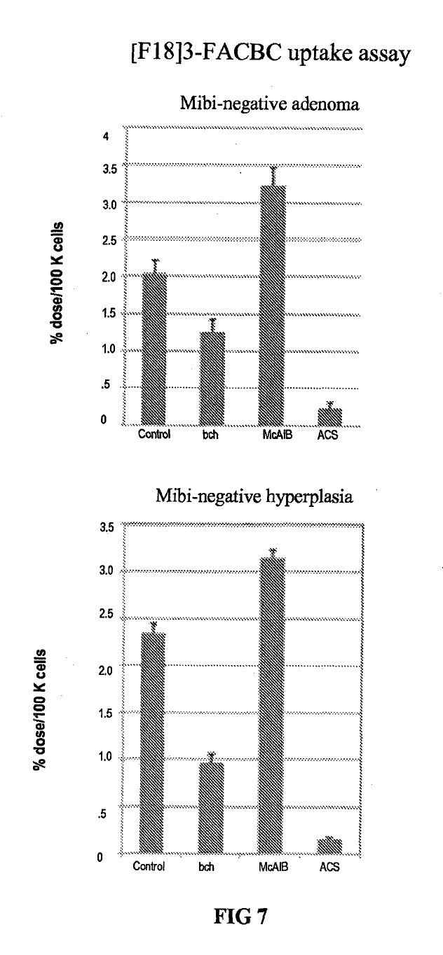

The specific targeting of FRs on freshly excised, non-cultured human PT adenoma cells was demonstrated by blocking FR receptors. Increasing amounts of PT adenoma cells (10 μl, 20 μl, and 70 μl of cell slurries) were incubated with 99mTc-EC20 in the presence or absence of cold folate solution, and the dose-dependent uptake of the radio-labeled compound was measured by gamma counting. 99mTc-EC20 uptake was significantly inhibited by pre-incubation with cold folate (for example, 3.4±0.4 not blocked vs. 1.9±0.2 blocked, % dose/20 μl tissue prep, p<0.05; 10.9±0.9 not blocked vs. 5.7±0.3 blocked, % dose/70 μl tissue prep, p<0.001) (FIG. 5).

1. A method comprising
a) administering a metal particle-folate-conjugate to a subject at risk of, suspected of, or diagnosed with a parathyroid tumor;
b) exposing an area suspected of containing the parathyroid tumor of the subject to a magnetic field and a radio frequency pulse; and
c) detecting nuclear resonance frequencies in the area.

2. The method of claim 1 further comprising the step of creating an image from the detected nuclear resonance frequencies.

3. The method of claim 1 wherein the metal particle is an iron oxide nanoparticle.

4. A method comprising
a) administering a radioisotope-folate conjugate to a subject at risk of, suspected of, or diagnosed with a parathyroid tumor, and
b) detecting gamma rays in an area of the subject.

5. The method of claim 3 further comprising the step of creating an image from the detected gamma rays.

6. The method of claim 4 wherein the radioisotope is ^{99m}technetium.

7. A method comprising
a) administering a composition comprising a positron-emitting radionuclide to a subject at risk of, suspected of, or diagnosed with a parathyroid tumor, and
b) detecting photons moving in approximately opposite directions in an area of the subject.

8. The method of claim 4 further comprising creating an image from the detected photons.

9. The method of claim 7 wherein the a positron-emitting radionuclide is anti-1-amino-[¹⁸F]flurocyclobutane-1-carboxylic acid (anti-¹⁸F-FACBC).
